(19) United States
(12) Patent Application Publication
(10) Pub. No.: US 2013/0000635 A1
(43) Pub. Date: Jan. 3, 2013

(54) TRACHEAL STOMA DILATION APPARATUS AND METHOD OF MANUFACTURE

(75) Inventors: James Curley, Offaly (IE); Sean Morris, Roscommon (IE); Olaf Lally, Galway (IE); Alan Finneran, Tullamore (IE); Colette Breheny, Galway (IE)

(73) Assignee: Nellcor Puritan Bennett LLC, Boulder, CO (US)

(21) Appl. No.: 13/173,105

(22) Filed: Jun. 30, 2011

(51) Int. Cl. A61M 16/04

(52) U.S. Cl. 128/200.26

(57) ABSTRACT

A tracheal dilator system and method of manufacture are provided, suitable for dilating a passageway into a patient airway. In one embodiment, a tracheal tube is provided. The tracheal tube includes a dilator retrieval tip disposed on a distal end of a tracheal tube cannula. The dilator retrieval tip is configured to retrieve a tracheal dilator configured to enter through the distal end of the tracheal tube and exit through a proximal end of the tracheal tube.

### BACKGROUND

The present disclosure relates to a tracheal dilation techniques, and more particularly to a tracheal dilation via a dilation cannula structure.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide range of applications exist for artificial ventilation, which may call for the use of tubes that are inserted into a patient. Such tubes may include endotracheal tubes, tracheal tubes, and so forth. In the latter case, the tubes are typically inserted into an opening or stoma formed in the neck and trachea of the patient. In both cases, the tubes may be used for artificial ventilation or for assisting patient ventilation. The stoma is typically formed either surgically, through a procedure such as a cricothyroidotomy, tracheostomy, or through a micro-surgical procedure such as percutaneous dilation. Cricothyroidotomy requires the use of a surgical team working in a sterilized environment to create an opening in the cricothyroid membrane, thus providing access to the patient's airway. The procedure typically involves the cauterizing of blood vessels, and typically has the patient undergoing general anesthesia.

Percutaneous dilation entails using an instrument, such as a needle or a scalpel, to make a small opening between the tracheal rings on a frontal or anterior region of the patient's neck. The needle or scalpel may then be inserted through the opening in the tracheal rings to allow a passageway into the patient's airway. A dilator, such as a curved cone shape dilator, similar to a horn, with increasing diameter from a distal tip to a proximal base, may then be pushed inwardly towards the trachea. As the dilator penetrates the stoma, the increasing diameter of the dilator may gradually expand the stoma until a desired size is reached, suitable for the insertion of the tracheal tube. However, the stoma may be breached to a size larger than a tracheal passageway for the tracheal tube, which may result in complications. Additionally, the breach may cause tears and scars in the frontal neck region.

### SUMMARY

The present disclosure provides a novel tracheal tube having a tube cannula suitable for use with forward and/or reverse dilation techniques, such as dilation techniques used in tracheostomy. The tracheal tube cannula may act as a retrieval guide for an expanding dilator, including expanding dilators having inflatable cuffs useful in forward and/or reverse dilation of the stoma. For example, the tracheal tube cannula described herein may include a distal tip (i.e., dilator retrieval tip) useful in guiding the dilator cuff into the tube cannula during the retrieval of the dilator from a patient's tracheal walls. The tracheal tube cannula's dilator retrieval tip may include features that enable the outwardly removal of the dilator's cuff from the tracheal wall by minimizing or eliminating an interference or “bunching” of the cuff against the dilator retrieval tip and the tracheal walls. Additionally, the dilator retrieval tip may include features that enable the insertion of the tracheal tube inwardly towards the trachea, thus placing the distal tip in a location suitable for providing respiratory support to a patient with minimal effort and trauma.

Advantageously, tracheal tube described herein may be used in combination with a reverse dilator having an expanding section, such as the reverse dilator disclosed in U.S. patent application Ser. No. 13/118,718 to James Curley, et al., filed on May 31, 2011, and entitled “REVERSE TRACHEAL STOMA DILATION METHOD AND APPARATUS,” which is hereby incorporated by reference for all purposes as if fully set forth herein. In one example, the reverse dilator may be inserted into the patient airway through the tracheal walls, and a resizable distal section of the dilation expander may then be enlarged or inflated. Accordingly, the dilation of the stoma may be performed beginning from an interior wall of the patient's airway rather than from an exterior neck region. The tracheal tube cannula described herein may then be inserted into the tracheal wall, using the reverse dilator as an insertion guide. Indeed, the tracheal tube cannula may have and inner diameter (ID) sized larger than an outside diameter (OD) of the reverse dilator, useful in enabling the insertion of the cannula following the outside walls of the reverse dilator. The reverse dilator cuff may then be deflated and the reverse dilator may be more easily removed by “sliding” the reverse dilator outwardly through the interior of the cannula, with minimal or no “bunching” of the reverse dilator cuff. The tracheal tube may then be used to provide ventilation support. Forward dilators may also be used with the tracheal tube cannula disclosed herein, as described in more detail below. By providing for a tracheal tube cannula enabling a more efficient removal of forward and/or reverse expanding dilators, the stoma opening may more closely conform to the tracheal tube outside walls.

In accordance with one embodiment, a tracheal intubation system having a tracheal tube is provided. The tracheal tube includes a dilator retrieval tip disposed on a distal end of a tracheal tube cannula. The dilator retrieval tip is configured to retrieve a tracheal dilator configured to enter through the distal end of the tracheal tube and exit through a proximal end of the tracheal tube.

In a similar arrangement, a tracheal tube includes a cannula configured to be disposed inside of a trachea. The tracheal tube further includes a dilator retrieval tip having a first portion configured to retrieve a tracheal dilator configured to enter through a distal end of the cannula and exit through a proximal end of the cannula.

Also provided is a method for manufacturing a tracheal tube. The method includes forming a cannula having a dilator retrieval tip disposed on a distal end of the cannula. The dilator retrieval tip comprises a portion configured to retrieve a tracheal dilator adapted to enter through the distal end of the tracheal tube and exit through a proximal end of the tracheal tube.

### BRIEF DESCRIPTION OF THE DRAWINGS

Advantages of the disclosed techniques may become apparent upon reading the following detailed description and upon reference to the drawings in which:

FIG. 1 is a sectional view of a patient's tracheal region and an insertion of a percutaneous needle;

FIG. 2 is a sectional view of a guide wire and the percutaneous needle inserted into the tracheal region of FIG. 1;

FIG. 3 is a sectional view illustrating an embodiment of a reverse dilator disposed in a tracheal region;

FIG. 4 is a sectional view of the same arrangement of FIG. 3, illustrating an inflated dilator cuff disposed on a distal portion of the reverse dilator;

FIG. 5 is a detail sectional view of the reverse dilator of FIG. 4 abutting an interior tracheal wall taken within arc 5-5;

FIG. 6 is a detail sectional view of the reverse dilator of FIG. 5 positioned inside a tracheal airway;

FIG. 7 is a detail sectional view of an embodiment of a tracheal tube having a dilator retrieval tip coupled to the reverse dilator of FIG. 6;

FIG. 8 is a detail sectional view of the dilator retrieval tip of FIG. 7;

FIG. 8a is a detail section view of a curved portion of the dilator retrieval tip of FIG. 8 taken within arc 8a-8a;

FIG. 9 is a detail section view of an embodiment of a conical dilator retrieval tip;

FIG. 9a is a detail section view of a rounded edge of the dilator retrieval tip of FIG. 9 taken within arc 9a-9a;

FIG. 10 is another detail section view of the tracheal tube of FIG. 7 coupled to the reverse dilator of FIG. 6;

FIG. 11 is a detail section view of a tracheal tube disposed inside an airway;

FIG. 12 is a section view illustrating an embodiment of a forward dilator disposed in a tracheal region;

FIG. 13 is a detail section view of the forward dilator of FIG. 12 taken within arc 13-13; and

FIG. 14 is a detail section view of the forward dilator of FIG. 13 coupled to the tracheal tube of FIG. 7.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

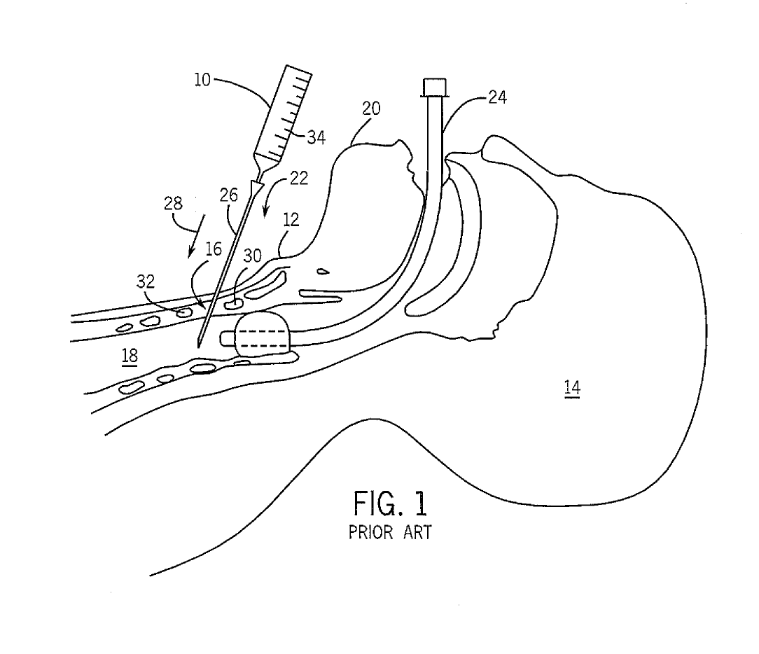

FIG. 1 is a sectional view illustrating a placement of a percutaneous needle 10 in a trachea 12 of a patient 14. By inserting the percutaneous needle 10 into the trachea 12, an initial opening or tracheal passageway 16 into an airway 18 is created, suitable for dilation. As depicted, the patient 14 is disposed in a supine position, with a chin 20 slightly elevated. In certain circumstances, a rostal traction on the tracheal 12 may be applied so as to gain neck hyperextension and better access to a frontal neck region 22. General or local anesthesia may be used (e.g., 1% lidocaine solution) to dull or eliminate any discomfort during the dilation procedure. Additionally, the patient 14 may be intubated, such as by using an endotracheal tube 24. Indeed, the systems and methods disclosed herein enable a dilation procedure with artificial respiration kept in situ. It is also to be noted that the systems and methods disclosed herein enable dilation without artificial respiration support (e.g., without the endotracheal tube 24).

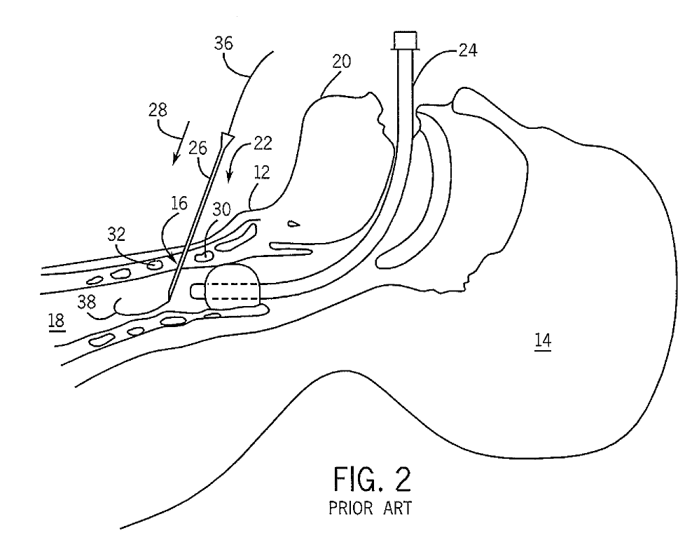

As depicted, a cannula 26 of the percutaneous needle 10 may be inserted in a direction 28, and enter the trachea 12 between a first 30 and a second 32 tracheal rings. As the percutaneous needle 10 is advanced in the direction 28, an aspiration of air through the needle 10 may indicate that the needle 26 has reached a desired position inside of the patient airway 18. Other methods useful in verifying that the cannula 26 is in the desired position may be used, such as a bronchoscopial survey, an ultrasound survey, and the like. It is also to be noted that other instruments may be used in creating the initial passageway 16 through the trachea 12. For example, a scalpel may also be used to provide a vertical or horizontal slit passageway 16 into the trachea 12. By using minimally invasive techniques to breach the trachea 12, scarring and other unsightly neck trauma may be minimized or avoided. Likewise, major bleeding during the dilation procedure may be eliminated. Once a clinician has verified that the needle cannula 26 has reached the desired position inside the airway 18, a body 34 of the needle 10 may be removed. A guide wire, such as a J-tip guide wire, may then be inserted through the cannula 26 of the needle 10, as described in more detail below with respect to FIG. 2.

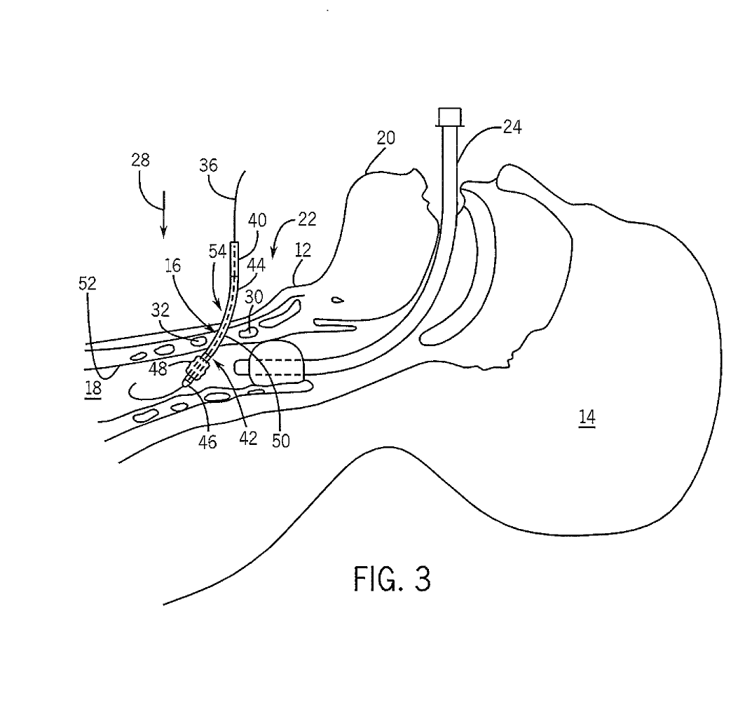

FIG. 2 is a sectional view depicting the insertion of a J-tip guide wire 36 into the patient's airway 18. Because the figure contains like elements found in FIG. 1, these elements are denoted using like reference numbers. As illustrated, the guide wire 36 is disposed inside of the needle cannula 26 and inserted so that a generally curved tip 38 is positioned inside the patient's airway 18. Using a guide wire, such as the J-tip guide wire 36, may enable a more efficient insertion of the dilation systems described herein. However, the dilation systems described herein may also be inserted into the trachea 12 without the use of any type of guide wire. When the J-tip guide wire 36 is used, the curved tip 38 may cause less trauma because the curved portion of the tip 38 is less likely to puncture the patient airway 18. That is, the curved tip 38 may prevent a “poking” or dagger effect. Once the curved tip 38 is inside the airway 18, the clinician may insert the guide wire 36 into a hollow shaft of a dilator, and then “slide” the dilator over the guide wire 36 to position a dilator partially inside of the patient airway 18, as depicted in FIG. 3.

FIG. 3 is a sectional view illustrating a reverse dilator 40 having a distal portion 42 positioned inside of the patient airway 18. As mentioned above, the guide wire 36 may be disposed inside a shaft 44 of the reverse dilator 40. The reverse dilator 40 may then be “slid” in the direction 28 over the guide wire 36, thus following the contours of the guide wire 36 and entering the patient airway 18. In certain embodiments, a “punch” dilator having a diameter larger than the guide wire 36, but smaller than a diameter of the shaft 44, may be used to dilate the passageway 16 prior to the insertion of the reverse dilator 40. To aid in the insertion into the airway 18, the reverse dilator may include a generally conically-shaped distal tip 46. Additionally, the distal portion 42 of the reverse dilator 40 may include an inflatable balloon cuff 48 positioned upstream of the distal tip 46. As depicted, the cuff 48 is fully deflated during the insertion of the reverse dilator 40 to minimize an interference force between the passageway 16 and outside walls 50 of the dilator 40. It is to be noted that, in other embodiments, the distal portion 42 of the reverse dilator 40 may include a resizable section manufactured out of shape memory alloys (e.g., Nitinol) or expandable by other mechanical techniques. Indeed, the resizable section may include either an inflatable cuff 48 or a mechanically expandable section.

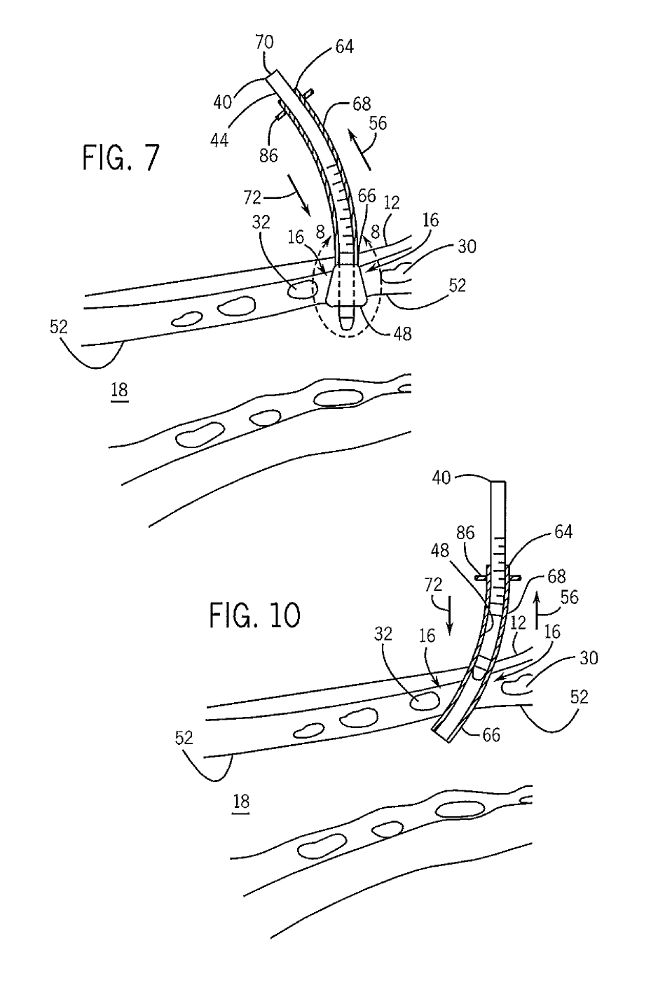

The reverse dilator 40 may be used to dilate the tracheal passageway 16 by initiating the dilation from an interior wall 52 of the airway 18 rather than by initiating the dilation from the exterior neck region 22 of the patient 14. Indeed, the reverse dilator 40 may be inserted into the airway 18 and then “pulled” outwardly from the airway 18 through the passageway 16. In this way, the passageway 16 is dilated from inside of the airway 18. It may be beneficial to dilate through the interior wall 52 of the airway 18 because the interior wall 52 may include softer tissues offering less resistance to dilation. Further, the interior wall 52 may include natural lubrication (e.g., airway moisture) useful in reducing a reverse dilation force. Additionally, scarring on the neck region 22 of the patient may be substantially reduced because the dilation breach occurs internal to the patient. Advantageously, the techniques disclosed herein enable a smoother withdrawal of the reverse dilator 40 by using a tracheal tube having a reverse dilation distal tip, as described in more detail below with respect to FIG. 7.

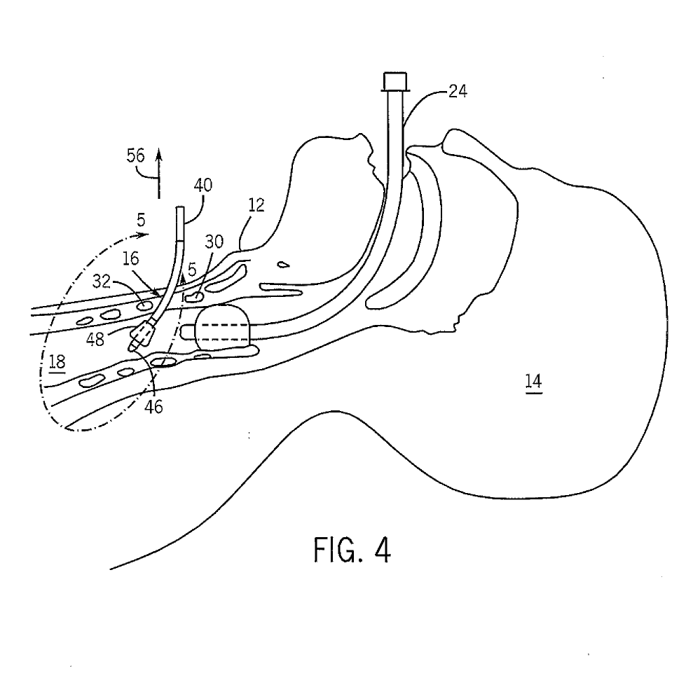

In one reverse dilation example, once the reverse dilator 40 is inserted into the desired region in the patient airway 18, the cuff 48 may then be partially or fully inflated, as depicted in FIG. 4. In this example, the partially or fully inflated cuff 48 may then be pulled outwardly through the passageway 16 in a direction 56, thus dilating the passageway 16 into a desired diameter. During emergency response procedures, it may be useful to fully inflate the cuff 40. In this way, a faster emergency response is enabled and the tracheal passageway 16 may be dilated to a diameter suitable for insertion of a variety of differently sized tracheal tubes. In other settings, such as an intensive care unit (ICU) setting, the clinician may select a dilator 40 including the cuff 48 of a desired diameter or inflate the cuff 48 to the desired diameter. The desired diameter is useful in accommodating a tracheal tube having a specific size. For example, tracheal tubes in a variety of sizes, such as between 2.5 to 10.5 mm ID may be dilated by inflating the cuff 48 to a desired cuff size. Other tube sizes, could, of course, be accommodated.

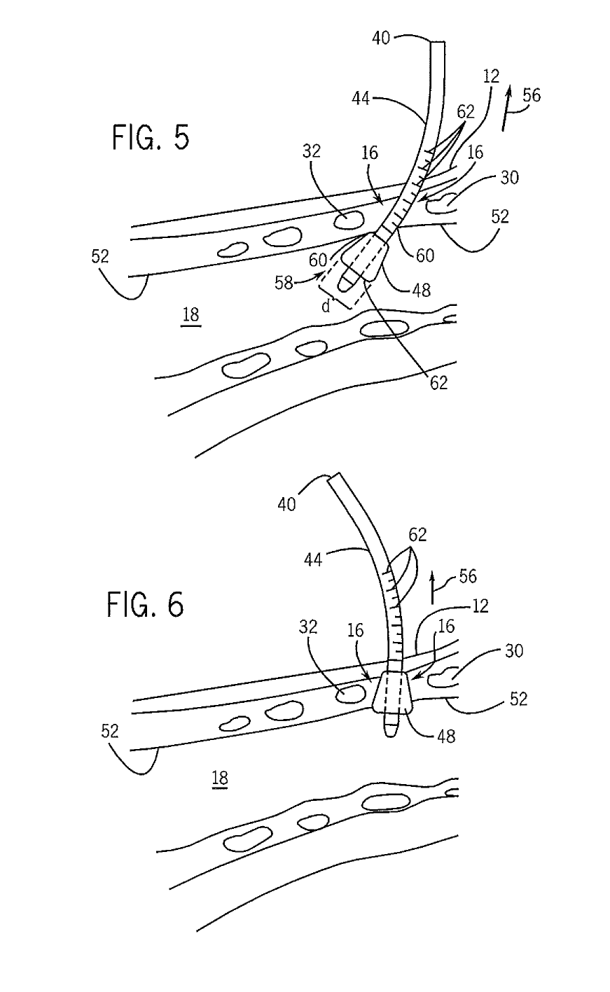

In one embodiment, a dilation cuff inflation system, such as a pump, may be used to provide a fluid flow (e.g., air flow, saline flow) to the cuff 48. The dilation cuff inflation system may use the ideal gas law, i.e., P×V=n×R×T, where P is a fluid flow pressure suitable for inflating a volume V at a temperature T based on the number of moles 11 of a gas and on the ideal gas constant R. Accordingly, the desired volume V for the cuff 48 may be provided by inflating the cuff 48 to the desired pressure P, taking into account temperature T, and incorporating the known values n and R, as depicted in FIG. 5. For example, the inflation P may be between about 15 cm H₂O and 20 bar. In another embodiment, the reverse dilator 40 may be manufactured in a variety of cuff 48 sizes, each cuff 48 sized to accommodate a tracheal tube of a given size (e.g., 2.5 to 14.5 mm OD). In this embodiment, the cuff 48 may be fully inflated so as to expand to its manufactured size.

FIG. 5 is a detailed sectional view illustrating an embodiment of the reverse dilator 40 of FIG. 4 with the cuff 48 inflated to a desired diameter d. As mentioned above, the desired diameter d may be derived by using the ideal gas law or the cuff 48 may be manufactured to be fully inflated to the diameter d. Further, the diameter d is generally derived to accommodate a tracheal tube having an OD approximately equal to d. Accordingly, the passageway 16 may be dilated to a size suitable for enabling the entry of a tracheal tube into the airway 18, while minimizing tissue trauma and scarring resulting from the dilation procedure. Indeed, the reverse dilator 40 may include other features, such as a generally conical shape 58 of the cuff 48, useful in minimizing the dilation effort and in lessening tissue trauma.

As depicted, the conical shape 58 increases in diameter, starting with a first diameter approximately equal to a diameter of the shaft 44 at a cuff attachment end 60 and ending in the diameter d at the base 62 of the cuff 48. As the reverse dilator 40 is pulled outwardly from the airway 18, the cuff attachment end 60 first makes contact with the interior wall 52 of the airway 18. By having a smaller diameter attachment end 60 as part of the cone shape 58, the cuff 48 may enable a smoother entry and dilation of the passageway 16 backwards through the interior wall 52 of the airway 18. Additionally, the cuff 48 may securely circumferentially encircle and “hug” the attachment end 60 to reduce trauma and insertion force. That is, the cuff 48 mating at the attachment end 60 may allow a smoother insertion through the interior wall 52 by eliminating protrusions or grooves at the attachment end 60. It is to be noted that other cuff shapes may be used, such as circular, square, and rectangular cuff shapes. It is also to be noted that, in other examples, the cuff 48 may be first fully deflated when penetrating into the interior wall 52. That is, the clinician may pull the reverse dilator 40 outwardly to position the fully deflated cuff 48 partially or fully in the passageway 16. The position of the cuff may be visually tracked by using markings 62 disposed on the shaft 44. Once the deflated cuff 48 is positioned and tracked by using the markings 62, the cuff 48 may then be inflated. The inflation of the cuff 48 inside of the passageway 16 may thus dilate the passageway 16. By inflating the fully deflated cuff 48 once the cuff 48 is inside the passageway 16, less pulling force may be used to position the reverse dilator 40 inside of the passageway 16.

FIG. 6 is a sectional view of an embodiment of the cuff 48 of the reverse dilator 40 disposed inside the passageway 16. In the depicted example, the cuff 48 may have been positioned in the passageway 16 either inflated (partially or fully), or fully deflated. If positioned fully deflated, the clinician may then have inflated the cuff 48. As mentioned above, the markings 62 may visually aid the clinician in tracking the position of the cuff 48 with respect to the passageway 16 to a desired position. In the illustrated position, the passageway 16 is now dilated at a size suitable for enabling the insertion of a tracheal tube, as depicted in FIG. 7.

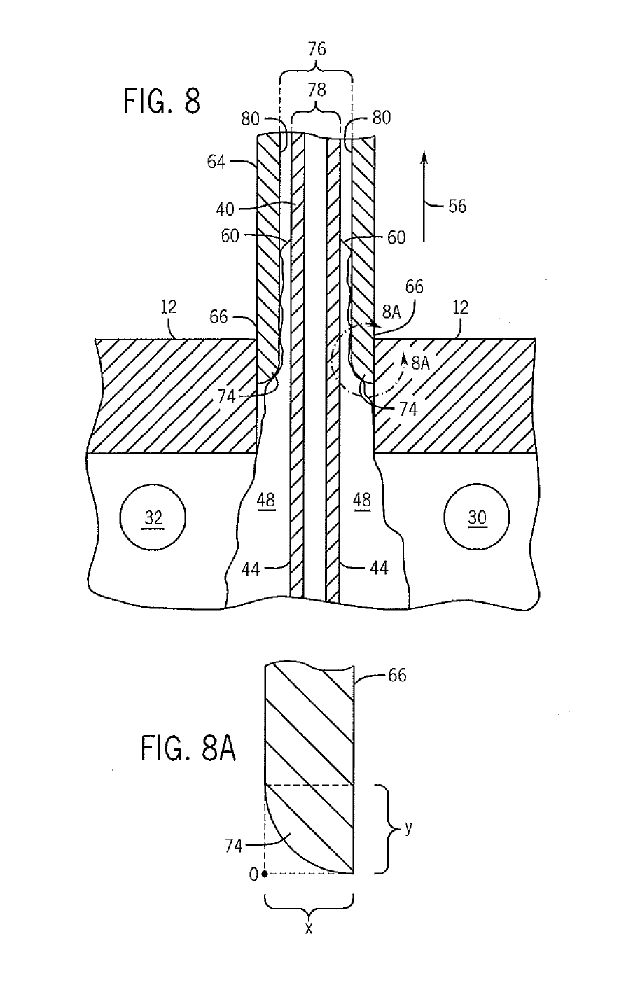

FIG. 7 is a sectional view on an embodiment of a tracheal tube 64 having a dilator retrieval tip 66 coupled to the reverse dilator 40. More specifically, the reverse dilator 40 has been disposed inside a cannula 68 of the tracheal tube 64. Indeed, an ID of the cannula 68 of the tracheal tube may approximately the same size or smaller than an OD of the shaft 44 of the reverse dilator 40. In this way, the reverse dilator 40 may be used as an insertion guide for the tracheal tube 64. For example, the clinician may insert a proximal end 70 of the dilator 40 into the cannula 68 and “slide” the tracheal tube 64 in a direction 72, following the outer walls of the shaft 44. The clinician may then insert the tracheal tube 64 into the dilated passageway 16. By using the dilator retrieval tip 66, the reverse dilator 40 may be more easily withdrawn from the tracheal passageway 16 and the tracheal tube 64 may be more easily placed into a desired position in the patient airway 18, as described in more detail with respect to FIG. 8 below.

FIG. 8 is a detail sectional view of the dilator retrieval tip 66 of FIG. 7 useful in more efficiently withdrawing a dilator, such as the reverse dilator 40, outwardly through the trachea 12 and in placing the tracheal tube 64 into a desired position in the airway 18. In the depicted embodiment, the dilator retrieval tip 66 includes a generally convex portion 74 defining a curved funnel-like shape suitable for receiving the shaft 44 and cuff 48 of the reverse dilator 40. The convex portion 74 enables the removal of the reverse dilator 40 through the cannula 68 of the tracheal tube 64, minimizing or eliminating “bunching” of the cuff 48 against the tracheal 12 walls. In the depicted example, as the reverse dilator 40 is outwardly retrieved in the direction 56, the cuff 48 attachment end 60 may come into contact with the convex portion 74 of the dilator retrieval tip 66. By including curved surfaces, the convex portion 74 may eliminate contact points having straight angle edges (i.e., 90° angle edges). Additionally, the convex portion 74 may aid in guiding the attachment end 60 of the cuff 48 into the cannula 68, providing for a minimal contact path that circumferentially encircles the cuff 48 and guides the cuff 48 through the cannula 68. As mentioned above, the cannula 68 may include an ID 76 slightly larger than an OD 78 of the reverse dilator 40, thus providing for an extraction path through inside walls 80 of the cannula 64 for the reverse dilator 68. In this manner, the reverse dilator 40 may be pulled outwardly through the trachea 12, while reducing any trauma that may have otherwise occurred during the removal of the reverse dilator 40. In one embodiment, the convex portion 74 may be approximately defined by an elliptical curve, such as a convex side of an astroid shape.

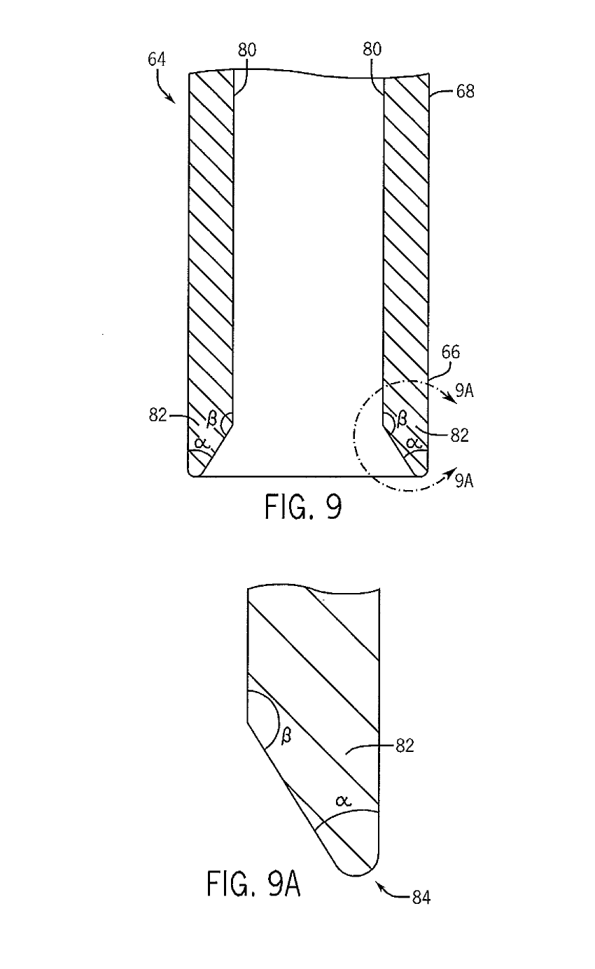

In the depicted embodiment of FIG. 8a, the convex portion 74 may be approximately defined by using one of the convex sides (e.g., first quadrant side) of an astroid shape with an origin o and having the parametric equation x=cos³ θ, y=sin³ θ, for 0≦θ≦2Π. Other convex-defining equations may be used to define the convex portion 74, including circular curves, ellipsoid curves, and more generally, equations defining sloping surfaces. In one embodiment, the tracheal tube 64, including the dilator retrieval tip 66, may be manufactured from a material such as a polyvinylchloride, a polyurethane, thermoplastic elastomers, a polycarbonate plastic, silicon, an acrylonitrile butadiene styrene (ABS), or a polyvinyl chloride (PVC). The convex portion 74 may be molded, overmolded, computer numerical control (CNC) machined, milled, or otherwise formed into the desired shape. Additionally, the dilator retrieval tip 66 may include embodiments having conical portions rather than convex portions, as described in more detail below with respect to FIG. 9.

FIG. 9 depicts and embodiment of the dilator retrieval tip 66 of the tracheal tube 64 having a generally conical portion 82 suitable for guiding a dilator, such as the reverse dilator 40, through to the inside of the cannula 68. In the depicted embodiment, the conical portion 82 may include a first angle α and a second angle β that combine to define a conical funnel shape for the conical portion 82. In certain embodiments, such as the embodiment depicted in FIG. 9a, the angle α may be between approximately 10° and 80°, while the angle β may be between approximately 100° and 170°. Smaller angles α and larger angles β may provide for a wider cone of entry, while larger angles α and smaller angles β may provide for a smaller cone of entry. Accordingly, the conical portion 82 may be manufactured at various angles α, β to suit different applications, such as applications having larger or smaller cuff 48 diameters, applications having larger or smaller cuff 48 wall widths, and applications having larger of smaller shaft 44 diameters. In one embodiment, such as the depicted embodiment, the conical portion 82 may include a rounded edge 84. The rounded edge 84 may further aid in reducing any trauma associated with the insertion of the tracheal tube 64 into the patient airway 18. Indeed, the rounded edge 84 (e.g., “bullnose” edge) may provide for a non-cutting, smooth edge suitable for guiding the reverse dilator 40 and cuff 48 into the inner walls 80 of the cannula 68. In this manner, the reverse dilator 40, including the cuff 48, may be more efficiently inserted into the cannula 68. By providing for a passageway out of the trachea 12 for the dilator 40, the dilator retrieval tip 66 may aid the clinician in more efficiently disposing the tracheal tube 64 at a desired position, as described in more detail with respect to FIG. 10 below.

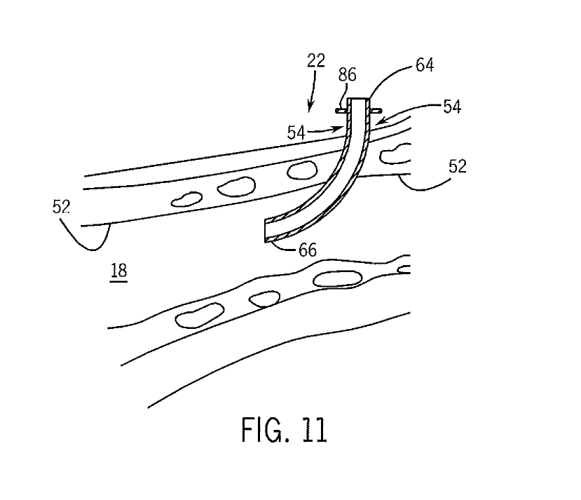

FIG. 10 is a sectional view of the tracheal tube 64 being disposed into the airway 18, while also enabling a more efficient removal of the reverse dilator 40. As mentioned above, the reverse dilator 40 may be inserted into the cannula 68 of the tracheal tube 64 and used to guide the tracheal tube 64 into the patient airway 18. The dilator retrieval tip 66 of the tracheal tube 64 may be used to more efficiently guide the dilator 40 and cuff 48 into the cannula 68. In the depicted example, the cuff 48 of the reverse dilator 40 has been fully deflated to reduce or eliminate an interference fit or friction between the reverse dilator 40 and the tracheal tube 64. The tracheal tube 64 may then be pushed inwardly in the direction 72 towards the passageway 16. Likewise, the reverse dilator 40 may then be pulled outwardly in the direction 56 away from the airway 18. For example, the tracheal tube 64 may be pushed inwardly until a set of flanges 86 approach approximately near the trachea 12. The reverse dilator 40 may then be fully removed from the cannula 68 of the tracheal tube 64, as depicted in FIG. 11.

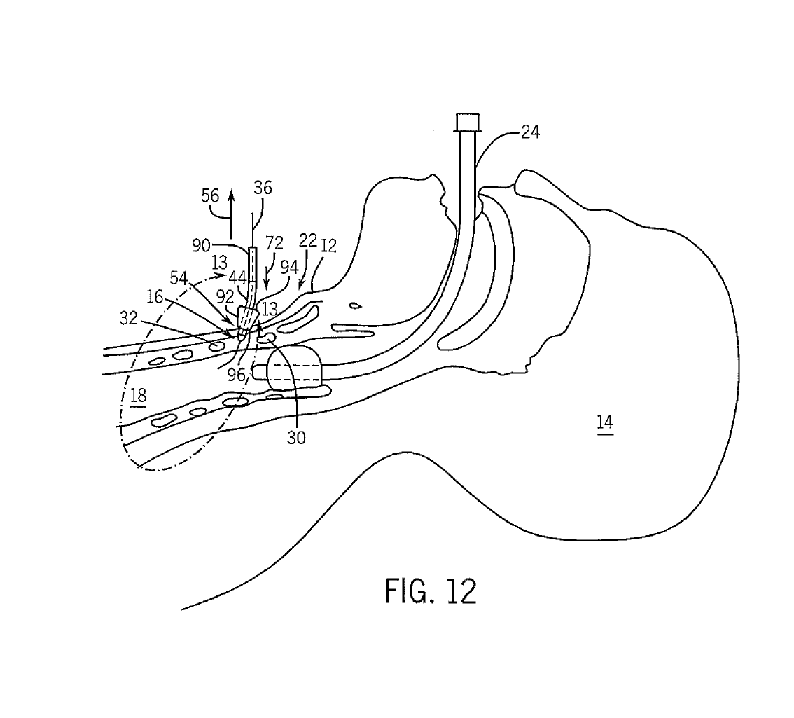

FIG. 11 depicts the tracheal tube 64 fully inserted into the airway 18 and ready to be used for respiratory support. Further, the reverse dilator 40, as shown in FIG. 10, has been removed to enable a connection of the tracheal tube 64 to, for example, a ventilator. By dilating the tracheal passageway from the inside the interior walls 52, unsightly tears or scars to the neck region 22 may be reduced. Likewise, trauma to patient tissue may also be reduced. Additionally, the diameter of the stoma 54 may more conformably fit the OD of the tracheal tube 64. The dilator retrieval tip 66 may also be used with forward dilator embodiments, as described in more detail below with respect to FIG. 12.

FIG. 12 depicts a forward dilator 90 having a cuff 92 suitable for dilating the trachea stoma in the forward direction 72, rather than a reverse, direction 56. In the depicted embodiment, the forward dilator 90 may follow the guide wire 36, as described above with respect to FIG. 3. In the depicted embodiment, the cuff 92 may be fully or partially inflated so as to initiate the dilation the stoma 54 beginning from the frontal neck region 22 o rather than from the airway 18. By initiating dilation from the outside of the neck, the clinician may more easily observe the expansion of the stoma and adjust dilation activities accordingly.

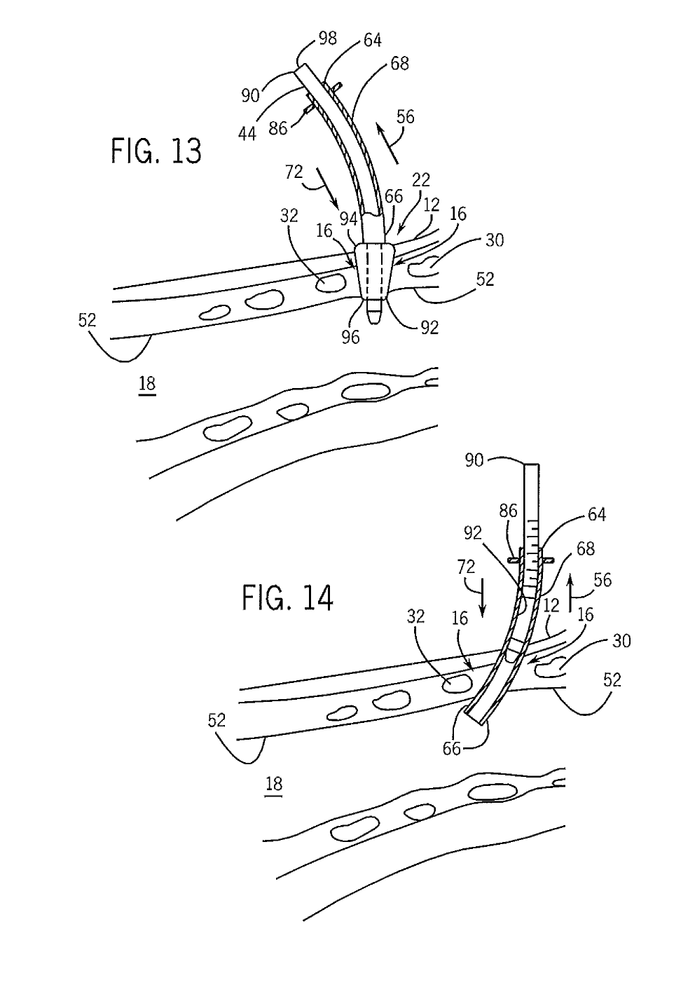

In one embodiment, such as the depicted embodiment, the cuff 92 may include a conical cuff attached to the shaft 44. The conical cuff 92 may further include a proximal base 94 having a larger diameter than a distal attachment end 96. Accordingly, the cuff 92 may more easily enter the stoma with the smaller diameter attachment end 96 being inserted first, followed by the remainder sections of the cuff 92. In other embodiments, the cuff 92 may be a circular cuff, a square cuff, or a rectangular cuff. Indeed, various cuff shapes may be used. Similar in function to the cuff 48 of the reverse dilator 40, the cuff 92 may be inflated and used to provide the passageway 16 by dilating the tracheal 12 walls from outside the neck. Once the cuff 92 has suitably dilated the tracheal 12 walls, then the tracheal tube 64 may be inserted by using the forward dilator 90 as a guide into the patient airway 18. The forward dilator 90 may then be retrieved through the cannula 68 of the tracheal tube 64, as described in more detail below with respect to FIG. 13.

FIG. 13 depicts an embodiment of the forward dilator 40 coupled to the tracheal tube 64 having the dilator retrieval tip 66. More specifically, the cuff 92 has been inflated and used to dilate the passageway 16 into the airway 18 from the frontal neck region 22. Following forward dilation, the forward dilator 90 may then be disposed inside the cannula 68 of the tracheal tube 64, as illustrated. As mentioned above, the ID of the cannula 68 of the tracheal tube 64 may approximately the same size or smaller than the OD of the shaft 44 of the forward dilator 90 to provide for a retrieval path of the forward dilator 90 through the interior of the cannula 68. By enabling the retrieval of the forward dilator 90 through the cannula 68, the tracheal tube 64 may provide for a more efficient dilation of the trachea 12 while minimizing or eliminating a “bunching” of the cuff 92. For example, the clinician may insert a proximal end 98 of the dilator 90 into the cannula 68 and “slide” the tracheal tube 64 in the direction 72, following the outer walls of the shaft 44 until the dilator retrieval tip 66 makes contact with the base 94 of the cuff 92. The cuff 92 may then be deflated, and the tracheal tube 64 may be positioned inside the airway 18.

The dilator retrieval tip 66 of the tracheal tube 64 may enable a smoother and more efficient removal of the forward dilator 90. Indeed, the dilator retrieval tip 66 may include the features described above with respect to FIGS. 7, 8, and 9, including the concave portion 74, the conical portion 82, and the curved edge 84, to provide for a guide into the cannula 68, while minimizing or eliminating “bunching” of the cuff 92. The forward dilator 90 may then be retrieved through the interior of the cannula 68, as depicted in FIG. 14.

FIG. 14 is a sectional view of the retrieval of the forward dilator 90 through the cannula 68. As mentioned above, the dilator retrieval tip 66 may more efficiently guide the forward dilator 90 and cuff 92 into the interior of the cannula 68 during removal of the forward dilator 90. In the depicted example, the cuff 92 of the forward dilator 90 has been fully deflated to reduce or eliminate an interference fit or friction between the forward dilator 90 and the tracheal tube 64. The tracheal tube 64 may then be pushed inwardly in the direction 72 towards the passageway 16. Likewise, the forward dilator 90 may then be pulled outwardly in the direction 56 away from the airway 18. For example, the tracheal tube 64 may be pushed inwardly until the set of flanges 86 approach approximately near the trachea 12. The forward dilator 90 may then be fully removed from the cannula 68 of the tracheal tube 64, and the tracheal tube 64 may be fully inserted into the airway 18, as depicted in FIG. 11. By providing for the dilator retrieval tip 66 useful in guiding the forward and the reverse dilators 90, 40 through the cannula 68, the tracheal tube 64 may enable a more efficient dilation that minimizes or eliminates “bunching” of the respective cuffs 48 and 92. Additionally, any trauma associated with forward or reverse dilation may be reduced.

What is claimed is:

1. A tracheal intubation system comprising:
a tracheal tube having a dilator retrieval tip disposed on a distal end of a tracheal tube cannula, wherein the dilator retrieval tip is configured to retrieve a tracheal dilator configured to enter through the distal end of the tracheal tube and exit through a proximal end of the tracheal tube.

2. The system of claim 1, wherein the dilator retrieval tip comprises a generally convex portion.

3. The system of claim 2, wherein the generally convex portion comprises a generally astroid shape, a generally elliptical shape, a generally circular shape, or a combination thereof.

4. The system of claim 1, wherein the dilator retrieval tip comprises a generally conical portion having a first angle α and a second angle β.

5. The system of claim 4, wherein first angle α comprises an angle between approximately 10° and 80°.

6. The system of claim 4, wherein the second angle β comprises and angle between approximately 100° and 170°.

7. The system of claim 1, wherein a distal end of the dilator retrieval tip comprises a rounded edge.

8. The system of claim 1, wherein the tracheal dilator comprises a reverse tracheal dilator configured to dilate a tracheal passageway leading into an airway from inside the airway.

9. The system of claim 1, wherein the tracheal dilator comprises a forward tracheal dilator configured to dilate a tracheal passageway leading into an airway from outside the airway.

10. The system of claim 1, wherein the tracheal dilator comprises a shaft and an expandable portion attached to the shaft, the expandable portion being configured to expand and contract, and wherein the shaft comprises an outer diameter smaller than an inner diameter of the tracheal tube cannula.

11. A tracheal tube comprising:
a cannula configured to be disposed inside of a trachea; and
a dilator retrieval tip having a first portion configured to retrieve a tracheal dilator configured to enter through a distal end of the cannula and exit through a proximal end of the cannula.

12. The tracheal tube of claim 11, wherein the first portion comprises a generally convex portion.

13. The tracheal tube of claim 12, wherein the generally convex portion comprises a generally astroid shape, a generally elliptical shape, a generally circular shape, or a combination thereof.

14. The tracheal tube of claim 11, wherein the first portion comprises an approximately conical portion having a first angle α and a second angle β.

15. The tracheal tube of claim 14, wherein first angle α comprises an angle between approximately 10° and 80°.

16. The tracheal tube of claim 14, wherein the second angle β comprises and angle between approximately 100° and 170°.

17. The tracheal tube of claim 11, wherein the dilator retrieval tip comprises a second portion distal of the first portion and having a rounded edge.

18. A method for manufacturing a tracheal tube comprising:
forming a cannula having a dilator retrieval tip on a distal end of the cannula, wherein the dilator retrieval tip comprises a portion configured to retrieve a tracheal dilator adapted to enter through the distal end of the tracheal tube and exit through a proximal end of the tracheal tube.

19. The method of claim 18, wherein the portion comprises a generally convex portion having a generally astroid shape, a generally elliptical shape, a generally circular shape, or a combination thereof.

20. The method of claim 18, wherein the portion comprises a generally conical opening having first angle and second unequal cone angles.
